(19) United States
(12) Patent Application Publication
(10) Pub. No.: US 2007/0167700 A1
(43) Pub. Date: Jul. 19, 2007

(54) Method for accurate in vivo delivery of a therapeutic agent to a target area of an organ

(76) Inventors: Norbert Rahn, Forchheim (DE); Jan Boese, Eckental (DE); Martin Kleen, Neunkirchen (DE)

Correspondence Address: SCHIFF HARDIN LLP;Patent Department, 6600 Sears Tower, 233 South Wacker Drive, Chicago, IL, 60606, US

(21) Appl. No.: 11/314,589

(22) Filed: Dec. 21, 2005

(51) Int. Cl. A61B 5/05

(52) U.S. Cl. 600/407

(57) ABSTRACT

In a method for accurately delivering a therapeutic agent to a target area of an organ of a living subject, such as for injecting stem cells into the myocardium of the heart, a 3D image, in which the target area and a delivery path thereto are visible, is obtained prior to delivery of the therapeutic agent. The 3D image is displayed, and a catheter is introduced into the subject and a real time positional indication of the catheter in the subject is obtained and incorporated into the displayed image, providing visual support for guiding the catheter to the target area. When the catheter is at the target area, the therapeutic agent is injected into the target area via the catheter. The distribution of the injected therapeutic agent relative to the target area is then monitored in the displayed image.

### BACKGROUND OF THE INVENTION

1. Field of the Invention

The present invention concerns a method for delivering a therapeutic agent to a target area of an organ in a living subject, and in particular to a method for accurately injecting stem cells into the myocardium of a heart.

2. Description of the Prior Art

Treating damaged myocardial areas of a heart by the injection of stem cells is an area of current biomedical research that appears promising. An advantage of the renewal of damaged myocardial areas by means of stem cells of an adult is that stem cells from the body of the patient can be propagated in cultures, and then re-supplied to the patient, without concerns about rejection thereof by the patient's own immune system.

Two possibilities currently exist for placement of the stem cells relative to the myocardium. One technique is to inject stem cells intra-arterially into coronary arteries that supply the damaged myocardium areas. Another known technique is interventional cardiology, wherein stem cells are directly injected into the damaged myocardial tissue using a catheter having a sheath or jacket in which an injection needle is inserted. Details regarding this use of interventional cardiology can be found at www.bioheartinc.com.

The most significant difficulty involved in using interventional cardiology for this purpose is to precisely guide the catheter (sheath) to a location close to the site of damaged myocardial tissue, and to subsequently guide the injection needle precisely to the damaged myocardial tissue site. A further difficulty is to provide a visualization of the stem cells themselves relative to the myocardial anatomy.

Similar problems exist in any context wherein a therapeutic agent must be accurately delivered to a target area of an organ of a living subject.

An overview of the current state of research in this area is provided in “Stem Cell Transplantation In Myocardial Infarction,” Lee et al, Review In Cardiovascular Medicine, Vol. 5, No. 2 (2004). Another overview of the current state of research in this area can be found at www.medreviews.com/pdfs/articles/RICM_{—}52_{—}82.pdf.

Several known techniques exist that have the goal of achieving accurate delivery or placement of stem cells to damaged myocardial areas. One such known technique is interventional MR (magnetic resonance), wherein the stem cells are given an MR-compatible “label” or “marker.” The labeled stem cells are injected into the damaged myocardial area by means of a catheter, under the supervision of interventional MR imaging.

Another technique is the surgical approach, wherein the stem cells are directly introduced into the damaged myocardial area in an open heart surgical procedure.

Another known technique is to use a navigation system without imaging. In this technique, scarred myocardial tissue can be visualized in a symbolic 3D representation of the myocardium using the NOGA navigation system available from Biosense-Webster. An injection needle catheter equipped with position sensors can be guided to the infarction scars for the purpose of stem cell injection.

After the stem cells have been injected, several known techniques exist for verifying or monitoring the location of the injected stem cells. If the stem cells have been marked with an MR-compatible label, the marked stem cells can then be imaged by magnetic resonance. Monitoring of transplanted cells is also possible using PET imaging. It is also known to undertake functional monitoring of the heart muscle activity by quantitative evaluation methods, such as monitoring the ejection fraction, the heart wall motion, etc. from images obtained using various imaging modalities, such as CT and MR. The improvement (or lack thereof) in the myocardial activity after the stem cell therapy can be assessed by means of a pre-therapy/post-therapy comparison.

### SUMMARY OF THE INVENTION

It is an object of the present invention to provide a method for delivering a therapeutic agent to a target area of an organ of a living subject that allows accurate delivery of the therapeutic agent to the target area, as well as allowing subsequent monitoring of the distribution of the delivered therapeutic agent with respect to the target area.

A further object of the present invention is to provide such a method that allows accurate in vivo delivery of stem cells to a damaged area of the myocardium of a heart.

The above objects are achieved in accordance with the present invention by a method wherein, prior to delivery of the therapeutic agent a 3D image of a portion of the subject is obtained and displayed, the displayed 3D image showing the target area and a delivery path to the target area. A catheter is introduced into the subject and a real-time positional indication of the catheter in the subject is obtained, with the real-time positional indication of the catheter being incorporated into the displayed 3D image, for use for guiding the catheter along the delivery path to the target area. While the positional indication of the catheter is still incorporated in the displayed image at the target area (after the catheter has reached the target area), the therapeutic agent is injected via the catheter into the target area. Since the target area is contained in the 3D displayed image, the distribution of the therapeutic agent relative to the target area can be monitored using the displayed 3D image.

The 3D image can be obtained, for example, using CT, MR, 3D ultrasound, PET or SPECT.

In an embodiment, the positional indication of the catheter in the subject is obtained using a navigation system that indicates the position of the catheter in the displayed 3D image, allowing a physician viewing the displayed 3D image to guide the catheter along the delivery path to the target area.

In a further embodiment, a monoplanar or biplanar x-ray system can be used to generate an x-ray image of the catheter and the surrounding environment in the subject, including the delivery path and the target area in a 2D x-ray image that is incorporated into the displayed 3D image.

In accordance with the invention, the entirety of the injection procedure and subsequent monitoring occurs, with the following items being displayed in a combined manner: catheter with injection needle, anatomy of the organ in question (such as the myocardium anatomy), the target area for the therapeutic agent injection (for example scarred, damaged myocardial tissue) and the therapeutic agent itself, for example, stem cells.

In the embodiment wherein stem cells are being injected as the therapeutic agent, in order to be able to track the injection and subsequent propagation of the stem cells during and immediately after the injection, the stem cell fluid is enriched with a contrast agent that allows at least a portion of the injected stem cells to be visualized in the displayed 3D image. A “contrast agent emulsion” in which the stem cells, the contrast agent and a fluid medium are ingredients, is injected. The contrast agent can be selected dependent on the imaging modality that is used to generate the 2D image for monitoring, that is mixed into the displayed 3D image. X-ray or MR contrast agents can be used for this purpose, for example.

### DESCRIPTION OF THE DRAWINGS

FIG. 1 schematically illustrates a displayed cardiac slice together with an indication of the position and orientation of an injection needle and/or a catheter sheath, in accordance with the principles of the present invention.

FIG. 2 is a schematic block diagram showing basic components of the inventive method.

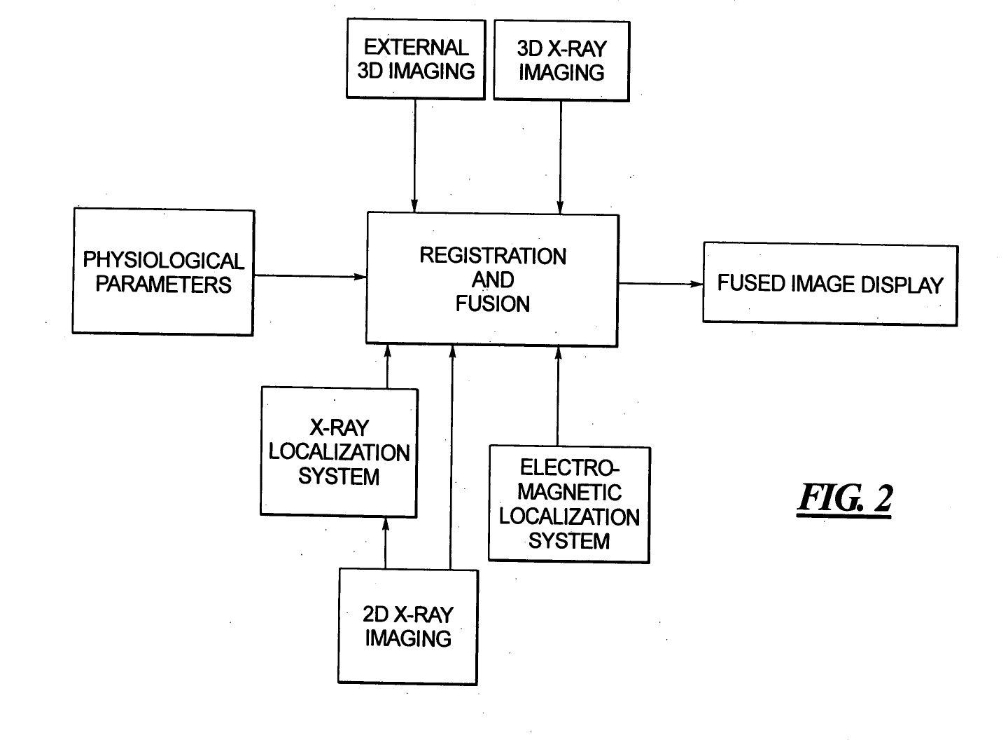

### DESCRIPTION OF THE PREFERRED EMBODIMENTS

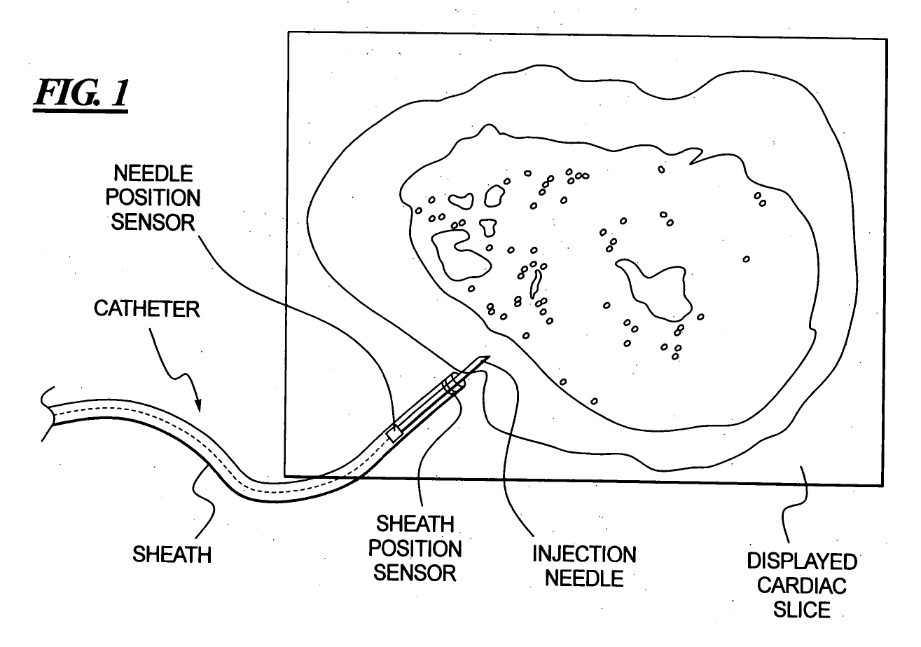

FIG. 1 schematically illustrates a displayed cardiac slice, in this case an axial slice, that is used in accordance with the invention to guide and monitor the administration of a therapeutic agent via a catheter. The catheter has a catheter sheath containing an injection needle via which a therapeutic agent, in liquid or emulsified form, can be delivered to a delivery site, in this case the myocardium. The injection needle and the sheath each have a position sensor that allows the respective positions of the sheath and the needle to be identified using a known navigation system.

In practice, the displayed cardiac slice is a 3D image that is obtained using a suitable imaging modality. The schematic representation of the displayed cardiac slice that is necessary for illustrative purposes in FIG. 1 will, in practice, be a conventional 3D medical image in which all of the features conventionally contained in, and identifiable in, such a 3D medical image will be present.

The 3D displayed cardiac slice shown in FIG. 1 is acquired using a medical imaging modality (CT, MR, 3D ultrasound, PET or SPECT) prior to beginning the interventional procedure to administer the therapeutic agent, and is thus referred to below as a pre-interventional 3D exposure. It is possible to obtain a number of pre-interventional 3D exposures with increasing image information content, in which case a superimposition (image fusion) of these multiple pre-interventional 3D exposures can be implemented, after the respective exposures are brought into registration with each other. Known techniques are available for such image fusion. Using a fused image formed by multiple pre-interventional 3D exposures is particularly useful when one or more of the pre-interventional 3D exposures contains additional information about the delivery site, such as information about a damaged myocardial tissue area. An image with such additional information can be acquired, for example, by PET or MR or CT with Late Enhancement.

In an embodiment of the invention wherein no additional imaging takes place during the interventional (delivery) procedure, the catheter and/or the injection needle can be visualized in the pre-interventional 3D image (image data) based on the known 3D position and orientation thereof obtained using a conventional navigation system, by means of the aforementioned position sensors.

As soon as the catheter sheath is at, or in the area of, the target site (lesion), guidance of the injection needle precisely to the target site (i.e., to damaged myocardial tissue) takes place in an image-supported manner by means of the needle position sensor attached to the needle, which allows the precise position of the needle in the pre-interventional 3D image to be visualized.

When the needle is precisely positioned at the target site, injection of the therapeutic agent takes place, such as injection of stem cells into a damaged myocardial tissue area.

After the delivery of the therapeutic agent, monitoring the distribution and accumulation of the injected therapeutic agent, such as injected stem cells in the myocardium, is implemented. If the intervention is implemented with the use of an imaging modality allowing imaging of the distribution of the injected therapeutic agent (for example, interventional CT or MR with the use of “labeled” stem cells), the distribution of the therapeutic agent (stem cells) in the relevant tissue (myocardial tissue, for example) is acquired and is superimposed on the pre-interventional 3D image. In such a superimposition, the cardiac/breathing phase in which the pre-interventional 3D image was acquired can be taken into consideration, so that the superimposed image is acquired at the same phase. Since the monitoring image will be a “real time” image, it most likely will encompass multiple cardiac cycles and respiration cycles. Known triggering techniques can be used to cause the monitoring image to be superimposed on the pre-interventional 3D image only when the monitoring image is at a phase that coincides with the phase shown in the pre-interventional 3D image. For example, the catheter position can be superimposed only at a time when such phase-coincidence exists, and the catheter position can be suppressed at other times.

If the distribution of the therapeutic agent in the relevant tissue area, as seen by the aforementioned monitoring, satisfies the therapeutic goal, the intervention is successfully ended. Otherwise, another delivery of therapeutic agent at a modified site can take place in the same manner as described above.

The 3D detection of the catheter using a navigation system can ensue with the use of miniaturized position sensors, for example operating electromagnetically, that are integrated into the catheter sheath and/or into the injection needle. A 3D-3D registration (for example, landmark-based) can then be implemented in a known manner between the coordinate system of the navigation system and the coordinate system of the 3D image data.

In a further embodiment of the inventive method, 2D and 3D x-ray imaging is undertaken during the intervention, and thus a navigation system (and the associated position sensors) are not used.

In this embodiment, a three-interventional 3D image is also acquired, as described above,

An interventional 3D x-ray image data set is obtained that represents an image in which the catheter and the tissue target area, such as myocardial tissue are visualized in 3D fashion. This 3D x-ray image data set can be re-acquired at one or more points in time during the intervention. Optionally, this 3D x-ray image data set can be superimposed with the pre-interventional 3D image data (after a 3D-3D registration). This is particularly useful when the pre-interventional 3D image data contain information about a damaged myocardial tissue area, for example scars that can be made visible with CT or MR imaging with Late Enhancement. During the intervention, continuous biplanar 2D x-ray imaging occurs. The catheter is visualized in real time in the 2D x-ray exposures, during the advancement of the catheter toward the target area. The x-ray exposures can be acquired in an ECG-triggered manner, and thus at a defined heart phase. This has the advantage of reducing the radiation dose to the patient, and allows conformity with the phase at which the pre-interventional 3D image data were obtained. Such real time biplanar 2D x-ray images with ECG triggering can be obtained using a system as described, for example, in U.S. Pat. No. 6,909,769, the teachings of which are incorporated herein by reference.

The 2D x-ray exposures can be superimposed with the pre-interventional 3D image data and/or the 3D x-ray image data set during the intervention. The current (real time) position of the catheter can be superimposed with the 3D image data, and the catheter thus can be guided to the target point with image support.

Optionally, the guidance can be undertaken based on the 3D position of the needle, if the needle is also provided with a marker allowing it to be visualized in the pre-interventional image data and/or in the 3D x-ray image data.

As soon as the catheter sheath is at a suitable location, the needle of the catheter is guided exactly to the target area, such as damaged myocardial tissue, with the support of the displayed image.

The therapeutic agent is then injected into the target area. If a stem cell emulsion enriched with contrast agent is injected, the distribution of the stem cells in the myocardial tissue can be tracked during or immediately after the injection using 2D or 3D x-ray imaging.

Even though injection may be implemented using ECG triggered 2D x-ray imaging, the injected contrast agent emulsion can be visualized, for example, with DSA imaging, with the same ECG triggering being used to ensure that images of the same heart phase are subtracted.

Alternatively, the 2D x-ray images in which the therapeutic agent is visualized can be superimposed with the pre-interventional image date or the interventional 3D x-ray image data. The stem cell distribution is thereby visualized in real time 3D image data.

It should be noted that the real time 2D x-ray images can, by off-line calibration of the biplanar C-arm system used to generate those images, be superimposed with the 3D x-ray image data (and with the pre-interventional 3D image data registered- with the 3D x-ray image data) without undertaking further registration. To compensate patient movements between the acquisition of the 3D image data and the superimposition, a 2D-3D image registration (using the known calibration as a starting value) can optionally be implemented. Should the current 2D x-ray image data be superimposed with the pre-interventional 3D image data, without a 3D x-ray image data set being acquired, the implementation of a 2D-3D registration between the 2D x-ray image and the pre-interventional 3D image data is necessary.

The detection of the 3D position/orientation of the catheter sheath, such as the catheter tip, can ensue using an electromagnetic position/orientation sensor that is integrated into the catheter sheath at or near the leading end (tip) thereof, and/or a similar sensor integrated into the catheter needle. The 3D position/orientation of the catheter is then visible in the pre-interventional acquired 3D image data or the 3D x-ray image data acquired during the intervention.

For visualizing the position and orientation of the catheter tip in the 3D x-ray image data acquired during the intervention (and thus in the pre-interventional 3D image data set registered therewith), no special registration is necessary as long as the spatial relation between the sensor cordinate system and the imaging modality coordinate system is known by an off-line calibration.

Alternatively, the 3D position/orientation of the catheter tip can be detected from two 2D x-ray images acquired from respectively different angulations. The detected 3D position then exists in the coordinate system of the x-ray system, and thus can be directly visualized in the 3D x-ray image data (and thus in the pre-interventional 3D image data registered therewith).

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for accurately delivering a therapeutic agent to a target area of an organ of a living subject, comprising the steps of:
prior to delivery of said therapeutic agent, obtaining a 3D image of a portion of an interior of said living subject and displaying said 3D image as a displayed image, said displayed image comprising said target area and a delivery path to said target area;
introducing a catheter into said subject and obtaining a real time positional indication of said catheter in said subject, and incorporating said real time positional indication of said catheter into said displayed image and therewith guiding said catheter along said delivery path to said target area;
while said positional indication of said catheter in said displayed image indicates said catheter is at said target area, injecting said therapeutic agent into said target area via said catheter, said target area being visible in said displayed image; and
causing the injected therapeutic agent to be visible in said displayed image, and monitoring distribution of said therapeutic image in said displayed image relative to said target area.

2. A method as claimed in claim 1 wherein said 3D image has a coordinate system associated therewith, and wherein the step of obtaining a real time positional indication of said catheter in said subject comprises providing said catheter with a sensor detectable with a navigation system having a coordinate system associated therewith, generating an indicator with said navigation system identifying a position of said sensor in the coordinate system of the navigation system, and bringing the coordinate system of the navigation system into registration with the coordinate system of the 3D image and superimposing said indicator indicating the position of said sensor with said 3D image in said displayed image.

3. A method as claimed in claim 2 wherein said catheter comprises a catheter sheath having a sheath tip, and wherein the step of providing said catheter with a position sensor comprises disposing a sheath position sensor at said tip of said sheath.

4. A method as claimed in claim 2 wherein said catheter comprises an injection needle, and wherein the step of providing said catheter with a position sensor comprises disposing a needle position sensor at said injection needle.

5. A method as claimed in claim 2 wherein said catheter comprises a catheter sheath having a sheath tip, and an injection needle projecting from said sheath tip, and wherein the step of providing said catheter with a position sensor comprises disposing a sheath position sensor at said tip of said sheath and disposing a needle position sensor at said injection needle.

6. A method as claimed in claim 1 wherein said 3D image has a coordinate system associated therewith, and wherein the step of obtaining a real time positional indication of said catheter in said subject comprises continuously obtaining a 2D x-ray image of said delivery path and said target area during introduction of said catheter into the subject, said 2D x-ray image having a coordinate system associated therewith, and bringing said coordinate system of said 2D x-ray image into registration with the coordinate system of said 3D x-ray image and superimposing said 2D x-ray image on said displayed image.

7. A method as claimed in claim 6 comprising obtaining said 2D x-ray image with a biplanar x-ray system.

8. A method as claimed in claim 6 comprising acquiring a 3D x-ray image at least of said target area and displaying said 3D x-ray image, said 3D x-ray image having a coordinate system associated therewith, bringing said coordinate system of said 2D x-ray image into registration with the coordinate system of said 3D x-ray image and superimposing said 2D x-ray image on the displayed 3D x-ray image.

9. A method as claimed in claim 8 comprising bringing the coordinate system of the 3D x-ray image into registration with the coordinate system of the 3D image, and superimposing said 3D x-ray image with said displayed image of said 3D image.

10. A method as claimed in claim 8 comprising obtaining said 3D x-ray image using a technique selected from the group consisting of computed tomography imaging with Late Enhancement and magnetic resonance imaging with Late Enhancement.

11. A method as claimed in claim 6 wherein the step of continuously obtaining a 2D x-ray image comprises obtaining an ECG of the subject, and triggering a plurality of successive 2D x-ray exposures at a same point in time in each of a plurality of successive cardiac cycles of the subject, using said ECG.

12. A method as claimed in claim 1 comprising acquiring said 3D image using an imaging modality selected from the group consisting of computed tomography, magnetic resonance and positron emission tomography.

13. A method as claimed in claim 1 comprising marking said therapeutic agent with a marking ingredient visible with an imaging modality, and obtaining said 3D image using said imaging modality, and wherein the step of monitoring distribution of said therapeutic agent comprises monitoring distribution of said ingredient in said displayed image.

14. A method as claimed in claim 1 wherein said target area is the myocardium of the heart of the living subject, and wherein the step of injecting said therapeutic agent into said target area comprises injecting stem cells into the myocardium.

15. A method as claimed in claim 14 comprising providing an emulsion of said stem cells and an ingredient visible in an imaging modality with which said 3D image is obtained, and wherein the step of injecting said therapeutic agent into said target area comprising injecting said emulsion into they myocardium.
